(19) United States
(12) Patent Application Publication
(10) Pub. No.: US 2019/0281944 A1
(43) Pub. Date: Sep. 19, 2019

(54) Disposable hair care member and method of using the same

(71) Applicant: Shih-Ling HSU, Tainan City (TW)

(72) Inventor: Shih-Ling HSU, Tainan City (TW)

(21) Appl. No.: 16/153,793

(22) Filed: Oct. 7, 2018

(30) Foreign Application Priority Data
Mar. 16, 2018 (TW) 107109183

(51) Int. Cl. A45D 19/16; A61K 8/02; A61Q 5/02

(52) U.S. Cl. CPC A45D 19/16; A45D 20/04; A61Q 5/02; A61K 8/0208

(57) ABSTRACT

A disposable hair care member contains: a body made of non-woven fabric which is absorbent, and the body includes essences with hair care ingredients. When having hair care, the body is spread and is wrapped around user's hairs, then the body is heated by using an external heat source so that the user's hairs absorb the essences of the body, thereafter the body is discarded after having the hair care, thus having quick hair care and personal hygiene.

### FIELD OF THE INVENTION

The present invention relates to a disposable hair care member and a method of using the same which are capable of having quick hair care and obtaining personal hygiene.

### BACKGROUND OF THE INVENTION

When having hair care in house, chemical hair cream is applied onto user's hairs with hands, and the user's hairs are wrapped by a towel after being messaged, then the hair care is realized by way of a hair cap or a hair steamer, thus causing troublesome hair care. Furthermore, it takes a long time to heat the user's head in hair caring process, so it is uncomfortable for the user. A hairdresser can realize hair care for his/her customers in hair salon by using hair care apparatus, but such a hair care apparatus is not disposable and has to be washed and sterilized, thus having poor personal hygiene.

The present invention has arisen to mitigate and/or obviate the afore-described disadvantages.

### SUMMARY OF THE INVENTION

The primary aspect of the present invention is to provide a disposable hair care member and a method of using the same which are capable of having quick hair care and obtaining personal hygiene.

Further aspect of the present invention is to provide a disposable hair care member and a method of using the same which contains the body which includes essences with hair care ingredients so as to avoiding applying chemical hair cream or fluid on user's hairs.

Another aspect of the present invention is to provide a disposable hair care member and a method of using the same which are capable of having hair care in house easily by wrapping the body of the disposable hair care member on user's hairs.

### BRIEF DESCRIPTION OF THE DRAWINGS

FIG. 1 is a perspective view showing the assembly of a disposable hair care member according to a preferred embodiment of the present invention.

FIG. 2 is a schematic view showing the operation of the disposable hair care member according to the preferred embodiment of the present invention.

FIG. 3 is another schematic view showing the operation of the disposable hair care member according to the preferred embodiment of the present invention.

FIG. 4 is also another schematic view showing the operation of the disposable hair care member according to the preferred embodiment of the present invention.

FIG. 5 is also still another schematic view showing the operation of the disposable hair care member according to the preferred embodiment of the present invention.

FIG. 6 is another schematic view showing the operation of the disposable hair care member according to the preferred embodiment of the present invention.

FIG. 7 is also another schematic view showing the operation of the disposable hair care member according to the preferred embodiment of the present invention.

### DETAILED DESCRIPTION OF THE PREFERRED EMBODIMENTS

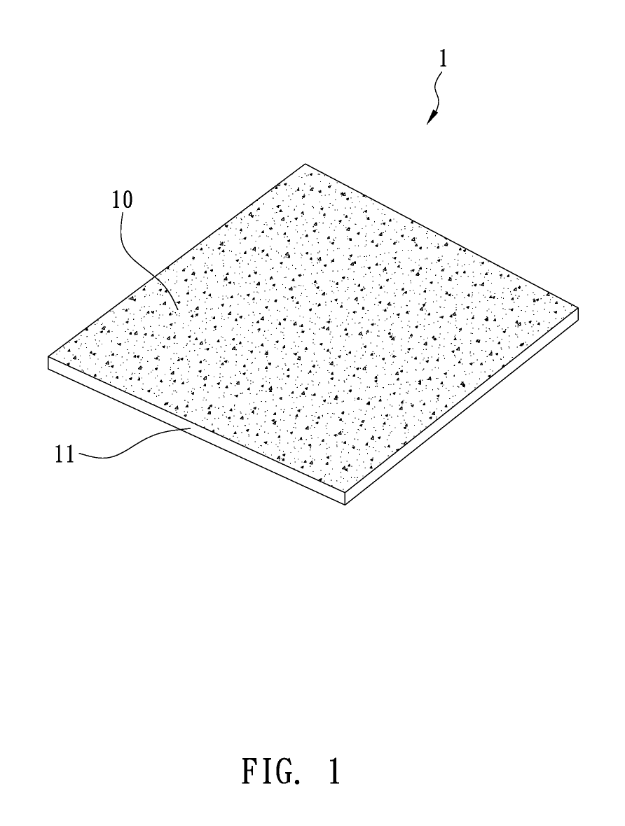

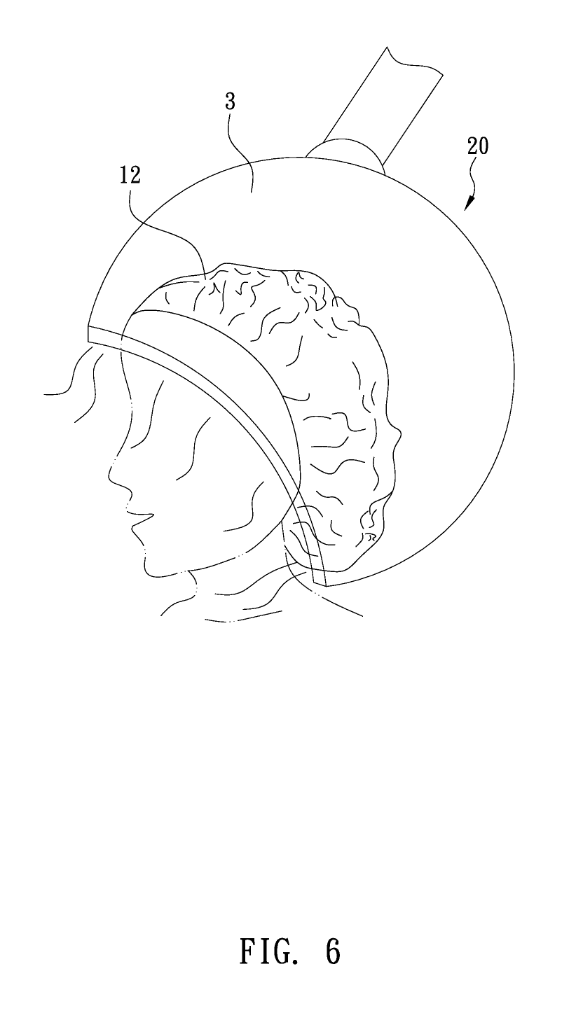

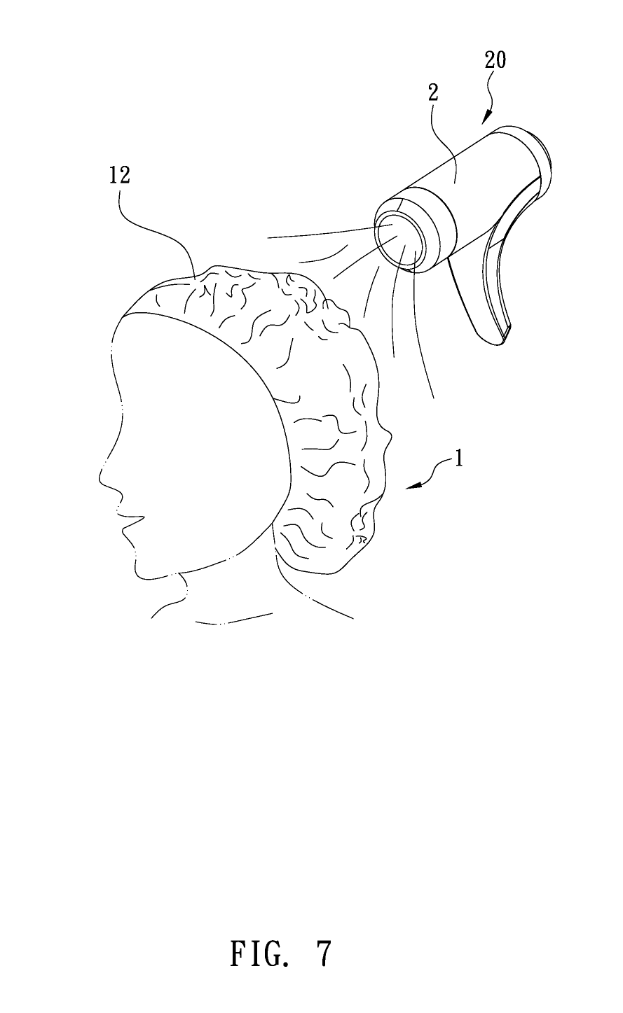

With reference to FIGS. 1-7, a disposable hair care member according to a preferred embodiment of the present invention comprises a body 1 made of non-woven fabric which is absorbent, and the body 1 includes essences 10 with hair care ingredients. When having hair care, the body 1 is spread and is placed on user's hairs 4, then the body 1 is heated by using an external heat source 20 so that the user's hairs 4 absorb the essences 10 of the body 1, thereafter the body 1 is discarded after having the hair care, thus having quick hair care and obtaining personal hygiene.

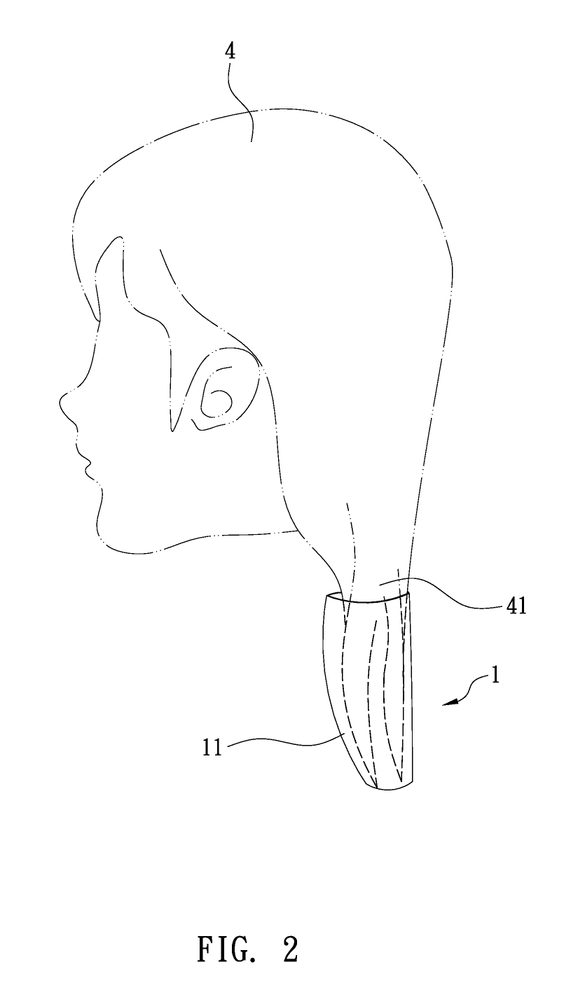

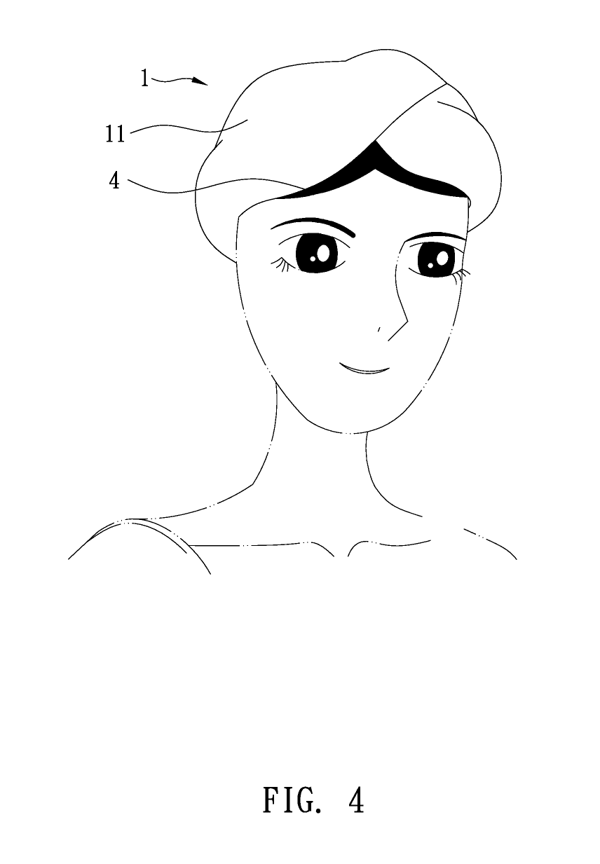

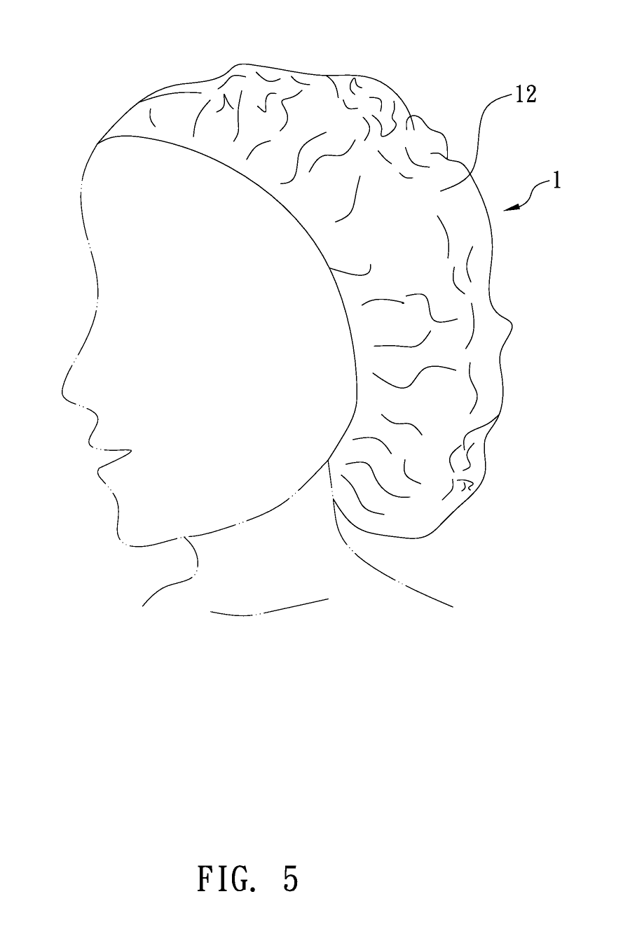

Referring to FIGS. 1, 2, 4 and 5, the body 1 is a flat film 11 or a hair cap 12, wherein the flat film 11 is flatly fixed on or is rolled on a part of the user's hairs 4. For example, the flat film 1 is rolled on the part of the user's hairs in a horse tail shape 41, as shown in FIG. 2. With reference to FIG. 4, when the flat film 11 is in a large size, it is wrapped around the user's hairs 4 completely. Furthermore, when the body 1 is the hair cap 12, it is covered around the user's hairs 4 completely, as illustrated in FIG. 5.

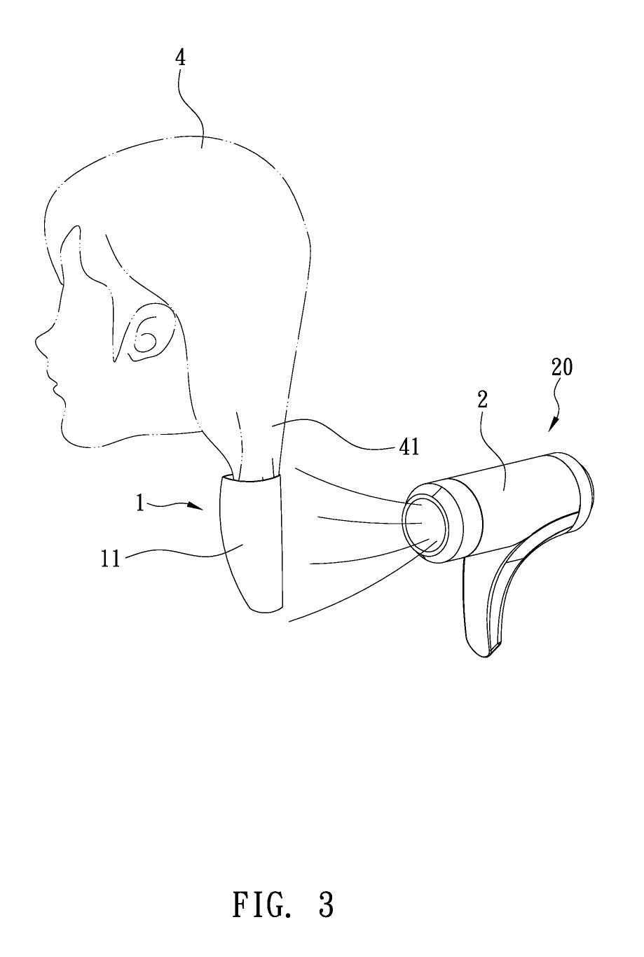

Referring to FIGS. 3, 6, and 7, the external heat source 20 is a hair dryer 2 or a hair steamer 3.

A method of using the disposable hair care member is executed by the user or a hairdresser, and the method comprises steps of:

Spreading the body 1, wherein when the body 1 is the flat film 11, it is flatly fixed on the part of the user's hairs 4 or is rolled on the part of the user's hairs 4 in the horse tail shape 41; when the flat film 11 is in the large size, it is wrapped around the user's hairs 4 completely; when the body 1 is the hair cap 12, it is covered around the user's hairs 4 completely;

Heating the body 1 by using the external heat source 20 so that the user's hairs 4 absorb the essences 10 of the body 1; and

Discarding the body 1 after having the hair care, thus having quick hair care and obtaining personal hygiene.

The body 1 is made of the non-woven fabric which is absorbent, and the body 1 includes the essences 10 with the hair care ingredients, such that the body 1 is spread and is wrapped around user's hairs 4 when having hair care, and the body 1 is heated by using the external heat source 20 so that the user's hairs 4 absorb the essence 10 of the body 1 quickly, and the body 1 is discarded, thus having quick hair care and obtaining personal hygiene.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention

What is claimed is:

1. A disposable hair care member comprising:
a body made of non-woven fabric which is absorbent, and the body including essences with hair care ingredients;
wherein when having hair care, the body is spread and is placed on user's hairs, then the body is heated by using an external heat source so that the user's hairs absorb the essences of the body, thereafter the body is discarded after having the hair care.

2. The disposable hair care member as claimed in claim 1, wherein when the body is a flat film, the flat film is flatly fixed on a part of user's hairs or is rolled on a part of user's hairs in a horse tail shape, wherein when the flat film is in a large size, it is wrapped around the user's hairs completely.

3. The disposable hair care member as claimed in claim 1, wherein when the body is the hair cap, the hair cap is covered around the user's hairs completely.

4. The disposable hair care member as claimed in claim 1, wherein the external heat source is a hair dryer or a hair steamer.

5. A method of using disposable hair care member comprising steps of:
(a). placing a body on user's hairs;
(b). heating the body by using an external heat source so that the user's hairs absorb essences of the body; and
(c). discarding the body after having the hair care, thus obtaining personal hygiene.

6. The disposable hair care member as claimed in claim 5, wherein when the body is a flat film, the flat film is flatly fixed on a part of user's hairs or is rolled on a part of user's hairs in a horse tail shape, wherein when the flat film is in a large size, it is wrapped around the user's hairs completely.

7. The disposable hair care member as claimed in claim 5, wherein when the body is the hair cap, the hair cap is covered around the user's hairs completely.

8. The disposable hair care member as claimed in claim 5, wherein the external heat source is a hair dryer or a hair steamer.
